(19) United States
(12) Patent Application Publication
(10) Pub. No.: US 2014/0377365 A1
(43) Pub. Date: Dec. 25, 2014

(54) SUSTAINED-RELEASE FORMULATION OF ROTIGOTINE

(71) Applicant: MAP Pharmaceuticals, Inc., Irvine, CA (US)

(72) Inventors: Libo WU, Mountain View, CA (US); Wiwik WATANABE, Union City, CA (US)

(21) Appl. No.: 14/308,007

(22) Filed: Jun. 18, 2014

Related U.S. Application Data
(60) Provisional application No. 61/837,006, filed on Jun. 19, 2013.

(51) Int. Cl. A61K 9/16; A61K 9/00; A61K 31/381

(52) U.S. Cl. CPC A61K 9/1652; A61K 9/1629; A61K 31/381; A61K 9/008
USPC 424/491; 424/489; 424/490; 424/493; 424/494; 424/497; 424/498; 424/45; 514/438; 549/75

(57) ABSTRACT

Provided herein are methods and compositions for producing formulations systemic delivery of dopamine agonists via the oral inhalation route. Specifically, provided herein are methods and compositions for a formulation of rotigotine that is suitable for administration via oral inhalation. Such methods and compositions are useful in the treatment or amelioration of one or more Parkinson's disease symptom(s).

### CROSS-REFERENCE

This application claims priority under 35 U.S.C. §119(e) from U.S. Provisional Application Ser. No. 61/837,006, filed Jun. 19, 2013, which is hereby incorporated by reference in its entirety.

### TECHNICAL FIELD OF THE INVENTION

The invention relates to new compositions and methods of treating Parkinson's disease symptoms. More specifically, the compositions and methods described herein are in the field of orally inhaled aerosol formulations. Specifically, compositions and methods that allow for the orally inhaled administration of rotigotine formulations are described.

### BACKGROUND OF THE INVENTION

Parkinson's disease is characterized by motor symptoms such as tremor, slowed ability to start and continue movements (bradykinesia), muscle rigidity, gait dysfunction and postural instability. All Parkinson's disease patients experience one or more of these symptoms, which progressively worsens with time. Researchers have identified that a degeneration of dopaminergic neurons in the substantia nigra area of the brain and degeneration of dopaminergic fibers as the primary pathophysiological mechanisms in Parkinson's disease. Additionally, researchers believe that other neurotransmitter systems such as serotonergic and glutamatergic systems are also involved in the disease process.

Rotigotine (5,6,7,8-tetrahydro-6-[propyl-[2(-thienyl)ethyl]amino]-1-naphthalenol, and its pharmaceutically acceptable salts have been known to be administered to patients through mostly transdermal delivery systems (see e.g., U.S. Pat. No. 7,413,747 and U.S. Pat. No. 6,884,434) and intranasal administration (see e.g., U.S. Pat. No. 7,683,040). Dopamine D2 agonists, such as rotigotine, may be effective agents in treating the symptoms of Parkinson's disease and other diseases for which an increase of dopamine levels may be beneficial, such as, but not limited to, restless leg syndrome (RLS). However, due complications from low bioavailability and the high systemic exposure of these dopamine D2 agonists, the development of safe and effective pharmaceutical formulations of dopamine D2 agonists, such as rotigotine, are needed.

Aerosols are increasingly being used for delivering medication for therapeutic treatment to the lungs. This type of pulmonary drug delivery depends on the subject inhaling an aerosol through the mouth and throat so that the drug substance can reach the lungs (i.e., oral inhalation). For drugs that are systemically active (e.g., the intended active site is not the lungs), inhalation delivery to the alveolar region of the lung is preferred.

Rotigotine has generally been formulated for transdermal delivery. However, there are consistency issues relating to transdermal delivery of rotigotine. Others have also described an intranasal formulation of rotigotine (U.S. Pat. No. 7,683,040). However, given the common impairment of motor control in Parkinson's disease patients, intranasal administration of rotigotine may be challenging and could require administration by a healthcare professional or in a hospital setting. Additionally, there would be complications due to consistency of dose through intranasal administration (e.g., insufflation) such as loss of the formulation on the nasal septum, where the formulation does not reach the intended nasal mucosa. Also, there may be significant loss of the formulation due to dose dripping down the throat and into the stomach. Oral inhalation delivery of dopamine D2 agonists such as rotigotine would overcome these difficulties and/or disadvantages.

There is a significant need for stable orally inhaled dopamine D2 agonists, such as rotigotine. The present invention satisfies this need.

### SUMMARY OF THE INVENTION

The invention encompasses methods and compositions of a pharmaceutical formulation comprising a dopamine agonist wherein the pharmaceutical formulation is suitable for administration by oral inhalation. In some embodiments, the dopamine agonist is rotigotine or a pharmaceutically acceptable salt thereof. In other embodiments, the formulation is administered by oral inhalation using a nebulizer, a pressurized metered dose inhaler or a dry powder inhaler. The metered dose inhaler can be breath-actuated and/or breath synchronized. The formulation may further comprise a propellant. The propellant can be 1,1,1,2-tetrafluoroethan, 1,1,1,2,3,3,3-heptafluoropropane or a mixture thereof. The formulation can be either solution-based or suspension-based. In some embodiments, the formulation is a suspension-based formulation and the size distribution of the rotigotine particles has a d₁₀ of about 0.5 mciron to about 1.0 micron, d₅₀ of about 1 micron to about 2 micron, and d₉₀ of about 2 micron to about 3 micron. In other embodiments, the size distribution of the rotigotine particles in the formulation has a d₁₀ of about 1 micron, d₅₀ of about 2 micron to 3 micron and d₉₀ of about 4 micron.

Another aspect of the invention describes compositions and methods of a pharmaceutical formulation comprising rotigotine or a pharmaceutically acceptable salt thereof, wherein the pharmaceutical formulation is suitable for administration by oral inhalation and wherein the formulation is suitable for controlled-release or sustained release of rotigotine in the lungs after administration by oral inhalation. In one embodiment, sustained-release or controlled-release of rotigotine is such that the rotigotine is physically encapsulated into a polymeric excipient. The polymeric excipient is selected from the group consisting of poly(lactic-co-glycolic acid), polylactic acid, polycaprolactone, cellulose, albumin, sodium hyaluronate, polyanhydrides, poly(vinyl acetate), polyethylene glycol, chitosan, hyaluronic acid, sodium alginate, starch, oligosaccharides, and polysaccharides. In another embodiment, the rotigotine is chemically conjugated to a carrier selected from the group consisting of a dendrimer, a hyperbranched polymer, polyethylene glycol, dextran, oleic acid, palmitic acid, and stearic acid. In other embodiments, the rotigotine is encapsulated in a solid lipid nanoparticle. In still other embodiments, the formulation is suitable for a pressurized metered dose inhaler and the formulation further comprises a propellant and an excipient selected from the group consisting of polyethylene glycol-polylactic acid copolymer, a sugar acetate, and polylactic acid.

### DETAILED DESCRIPTION OF THE INVENTION

This detailed description of the invention is divided into sections for the convenience of the reader. Section I provides definitions of terms used herein. Section II provides a description of methods and compositions of orally-inhaled dopamine agonists. Section III provides a description of oral inhalation delivery systems. Section IV discloses examples that illustrate the various aspects and embodiments of the invention.

### I. DEFINITIONS

Unless defined otherwise, all technical and scientific terms used herein have the meaning commonly understood by a person skilled in the art to which this invention belongs. As used herein, the following terms have the meanings ascribed to them unless specified otherwise.

“Active pharmaceutical ingredient” or “API” refers to active chemical(s) used in the manufacturing of drugs. Another term synonymous with API is “bulk drug substance”. It is understood that API refers to the active pharmaceutical ingredient including any and all appropriate salts, hydrates, solvates, polymorphs, prodrugs, ion pairs, and metabolites thereof.

“Colloid” refers to a chemical system composed of a continuous medium (continuous phase) throughout in which are distributed small particles (dispersed phase) that do not settle out under the influence of gravity. The particles may be in emulsion or in suspension.

“Drug composition” or “drug formulation” refers to a composition comprising at least one API and at least one additional composition.

“Excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Non-limiting examples of excipients include stabilizing agents, surfactants, surface modifiers, solubility enhancers, buffers, encapsulating agents, antioxidants, preservatives, nonionic wetting or clarifying agents, viscosity increasing agents, and absorption-enhancing agents.

“Hydrofluorocarbon” refers to hydrofluoroalkanes (HFAs). In recent years, HFAs have replaced chloroflurocarbons (CFCs) as propellants due to environmental issues concerning the impact of CFCs on the earth's ozone layer. Examples of hydrofluoroalkane propellants include of 1,1,1,2-tetrafluoroethane (referred to as HFA134a) and 1,1,1,2,3,3,3-heptafluoropropane (referred to as HFA 227).

“Oswalt ripening” refers to the thermodynamically-driven process based on the principle that larger particles are more energetically favored than small particles.

“Particulate API” refers to an API that is manufactured at a desired particle size or particles of a desired particle size range.

“QT Prolongation” refers to a prolonged period between the Q wave and the T wave in an electrocardiogram (heart's electrical cycle). In general, the QT interval represents electrical depolarization and repolarization of the right and left ventricles of the heart. QT prolongation can occur as a side-effect of certain medication(s) and is a biomarker for ventricular tachyarrhythmias and is a risk factor for sudden death.

“Solution” refers to a homogeneous mixture composed of only one phase. As applied to the invention, the API is dissolved in a suitable solvent or diluent to form a stable solution.

“Stabilized pharmaceutical formulation” refers to a pharmaceutical formulation that exhibits physical and chemical stability in which the physical and chemical composition characteristics of the formulation do not change significantly due to the effects of time and temperature.

“Surface modifier” refers to organic or non-organic pharmaceutically acceptable excipients that are typically added to a drug formulation to alter formulation performance. Such alterations in performance include reduction, minimization or elimination of aggregation or agglomeration of particle of a drug. Surface modifiers include, but are not limited to, polymers, low molecular weight oligomers, and surfactants.

“Suspension” refers to a chemical system composed of components in a medium where the components are larger than those comprising the medium. Components of a suspension can be evenly distributed, for example by mechanical means, however, the components will settle out of the medium under the influence by gravity.

“Unit dosage form” refers to a physically discrete unit suitable as unitary dosages for an individual, each unit containing a predetermined quantity of active material calculated to produce a desired therapeutic effect, in association with a suitable pharmaceutical carrier, diluent, solvent, or excipient. These unit dosage forms can be stored in suitable packaging in single or multiple unit dosages and may also be further sterilized and sealed.

It is to be understood that this invention is not limited to particularly exemplified drug particles, formulations, or manufacturing processes parameters as such, may vary. It is also to be understood that the technical terminology used herein is for the purpose of describing particular embodiments of the invention only, and is not intended to be limiting.

### II. METHODS AND COMPOSITIONS OF ORALLY-INHALED DOPAMINE AGONISTS

### Dopamine D2 Agonists

Because there are no proven disease-modifying therapies for Parkinson's disease, the primary goal of medical treatment is managing the multitude of motor and behavioral symptoms associated with the disease and improving patient's quality of life. L-dopa or levodopa is currently the “gold standard” for the management of motor symptoms of Parkinson's disease. L-dopa (L-3.4-dihydroxyphenylalanine) is the precursor to the neurotransmitters dopamine, norepinephrine and epinephrine. However, there are many adverse side-effects associated with chronic administration of levodopa.

One of the many symptoms of Parkinson's disease that many patients face is “freezing”. Freezing is the temporary, involuntary inability to move. Freezing may occur at any time and some patients are more prone to freezing than others. In many cases, patients may experience freezing of gait when the patient is due for the next dose of dopamine precursor therapy (e.g., levodopa), such a period is referred to as an “off period”. To alleviate freezing, current treatment calls for the increase of dopaminergic medication(s) in order to avoid the off period. However, because administration of dopaminergic medications are usually by oral therapy (e.g., pill or tablet), the time to wait for the medication to become bioavailable is quite lengthy (usually about 1-2 hours). Additionally, the administration of more dopaminergic medication such as levodopa may increase the side effects such as end of dose deterioration of function, on/off period oscillations, increase in freezing during movement, other motor response complications, drug resistance, dyskinesia, serotonin depletion, and dopamine dysregulation.

The ideal candidate for rescue treatment of Parkinson's disease symptoms should have a fast onset and short half-life; be effective in treating the Parkinson's off period; reduced nausea as to eliminate the need for an antiemetic; non-invasive and convenient route of administration; and minimal drug interactions (e.g., can be co-administered with levodopa).

The current rescue treatment for Parkinson's disease symptoms is apomorphine. Apomorphine is a morphine-derived is usually administered through injection. Administration through injection has the advantage of having a fast onset of action. Also, apomorphine has a short half-life and is effective in treating the off periods. However, its drawbacks are that injection is invasive and not convenient. Additionally apomorphine causes nausea, which requires co-administration with an antiemetic, and apomorphine may cause QT prolongation.

Dopamine agonists have been used for more than two decades as adjuncts to levodopa for patients suffering from levodopa-related motor response complications. Adjunct dopamine agonist therapy enables a lower dose of levodopa, which can ameliorate levodopa-induced side effects. The addition of a dopamine agonist can also help to extend the patient's “on” period and to relieve the effects of an off period. One such dopamine agonist is rotigotine. Currently rotigotine is available as a transdermal patch. However, drawbacks exist for transdermal delivery of rotigotine including dosing issues and patient compliance. The present invention addresses both of these problems.

An orally inhaled rotigotine formulation may be advantageous as a rescue therapy for Parkinson's off periods. Rotigotine's relatively short half-life is ideal for rescue therapy. Rotigotine is a dopamine D2 agonist and is a proven therapy for managing motor symptoms associated with Parkinson's disease. Rotigotine is also highly lipophilic which makes it suitable for rapid penetration through the lung epithelial barrier and the blood brain barrier. Additionally, there seems to be less adverse side-effects associated with rotigotine than other dopamine agonists such as ropinerole and pramipexole.

Systemic delivery via the oral inhalation route (and thereby through absorption in the lungs into systemic circulation) provides several advantages when the primary intended site of action of the drug is the brain. One advantage is the very rapid absorption by the lung and delivery into systemic circulation. Once absorbed by the lungs, the drug will enter into the pulmonary artery and then to the carotid artery to the brain. Once in the brain, the drug can cross the blood-brain barrier and be delivered to the intended site of action. This targeted delivery to the brain avoids first pass metabolism and avoids any enzyme degradation that may occur. Because the brain (via the carotid) is one of the first major organ that is engaged via this route of systemic circulation, oral inhalation also can minimize potential systemic side effects and may lower the dose required for efficacy in a subject.

Another advantage for an orally inhaled rotigotine formulation is the relatively fast onset of action for drugs that are administered to the lungs for systemic delivery to brain (one site of action). Compared to oral administration through a pill or tablet which has an onset of action of between 1-2 hours, oral inhalation/pulmonary administration for systemic delivery to the brain has an onset of action usually of less than 20 minutes after administration. Because of the rapid onset of action achieved through pulmonary administration of systemically active drugs, this method of delivery is preferred for acute treatment of symptoms such as rescue from Parkinson's freezing event(s).

Also, unlike oral administration, pulmonary administration through oral inhalation bypasses the gastrointestinal tract and thus also avoids enzymatic degradation, problems with gastric stasis (in some diseases) and inconsistent absorption rates, giving the patient a more consistent delivery of the drug. Unlike IV or IM injection, pulmonary administration through oral inhalation is convenient, non-invasive, self-administrable and no hospitalization is required.

In some embodiments, the orally inhaled active pharmaceutical ingredient (API) is rotigotine. In other embodiments, the API formulation is a rotigotine maleate salt solution.

### Aerosol Formulations

Aerosol formulations of an API may be in either a suspension or a solution. Particulate active pharmaceutical ingredient (API) that are of an acceptable particle size for delivery to the lungs in a suspension aerosol formulation may be generated in a variety of manner. For illustrative purposes, API particles may be generated from the bulk API by attrition processes such as grinding, micronizing, milling or the like. API particles may also be generated through a multiphase precipitation process such as spray drying, solution precipitation, in situ precipitation, volume exclusion precipitation, supercritical extraction/precipitation, lyophilization, or the like. API particles for use in aerosols are generally manufactured to a size of about 0.05 microns to about 10 microns, of about 0.1 microns to about 5 microns, of about 0.5 microns to about 3 microns, and of about 1 micron to about 3 microns. In various embodiments, the active pharmaceutical ingredient has a particle size in the range of about 0.5 microns to about 3 microns. In other embodiments, the API has a particle size in range of about 1 micron to about 3 microns.

Aerosol solution formulation is less concerned with the particle size of the API. Bulk API may be used as long as the API forms a stable solution (i.e., no precipitate formation) in a suitable solvent. Although currently, the only solvent/cosolvent that has been approved by the Food and Drug Administration for use in oral inhalation formulation is ethanol, other potentially suitable solvents/cosolvents that may be used include propylene glycol, polyethylene glycol and water.

### Formulation for Oral Inhalation

The invention is directed to a pharmaceutical composition in unit dose form comprising rotigotine in an amount such that one or more unit doses are effective in the symptomatic treatment of one or more Parkinson's disease symptom(s) when administered to a patient. In some embodiments, the rotigotine is free base. In other embodiments, the rotigotine is a salt form. Suitable salt forms of rotigotine include, rotigotine glycolate, rotigotine lactate, rotigotine maleate, rotigotine palmitate, rotigotine pamoate, rotigotine propionate and rotigotine stearate.

Inhalation aerosols of drug formulation for delivery using a pressurized metered dose inhaler typically include excipients such as surfactants and other surface modifiers to increase the stability of the particles or to increase the deliverability of these drugs in an aerosol form. However, excipients such as surfactants and other surface modifiers have been associated with toxicity in the subject and other undesirable side effects. To avoid or minimize such toxicity problems, the drug formulation of the present invention is free of excipients such as surfactants and other surface modifiers whenever possible. To the extent that the exclusion of such surfactants and other surface modifiers is not possible, such surfactants and other surface modifiers should be included at the very lowest concentration while preserving their effects.

The drug formulation may include one or more active pharmaceutical ingredient in any appropriate amount (singularly or in aggregate). In some embodiments, the API(s) may be selected to be in a certain concentration in order to achieve a desired concentration(s) after delivery into the subject or patient. In other embodiments, the API(s) may be selected to be in a certain concentration to conform to a certain dosing regimen or to achieve a certain desired effect.

Stability of a solution-based formulation can be determined by a variety of methods. One such method is to measure precipitate formation (if any) over time in different temperature/humidity conditions. Precipitate formation depends on the API interaction with the solvent and with the propellant for solution-based formulations. In some embodiments, a stable aerosol formulation will not have precipitate formation after 1 week at room temperature. In other embodiments, a stable aerosol formulation will not have precipitate formation after 1 week at 4-8° C.

Another desired characteristic of a solution-based formulation is the measure of the mass median aerodynamic diameter (MMAD) of the droplet size emitted from the device. The aerosol performance of a solution formulation is dependent on various factors such as propellant type (makeup), amount of solvent/cosolvent, API concentration and the container closure system. Unlike a suspension-based formulation, the API particle size is not a decisive factor for MMAD of a solution-based formulation since the API is solubilized in the propellant/solvent/cosolvent mixture. For proper deposition of the emitted formulation in the lung epithelium, a preferred range of MMAD is required. In some embodiments, the MMAD of the emitted formulation is between 1 micron and 5 microns. In other embodiments, the MMAD of the emitted formulation is between 2 microns and 3 microns.

For suspension-based formulations, it is preferred to have a narrow particle size distribution to avoid Ostwald ripening, which is essentially a process where the large particles grow at the expense of smaller particles. Ostwald ripening is a thermodynamically-driven process based on the principle that larger particles are more energetically favored than small particles. Ostwald ripening can increase the particle size over time and thus deteriorate the aerosol performance of the formulation. The Ostwald ripening effect can be minimized by using a drug particle population with a narrow size distribution range. In suspension-based formulations, it is preferred to have a size distribution of d₁₀ of about 0.5 micron to about 1.0 micron, d₅₀ of about 1 micron to about 2 micron and d₉₀ of about 2 micron to about 3 micron. In some embodiments, it may be preferred to have a size distribution of d₁₀ of about 1.0 micron, d₅₀ of about 2 micron to about 3 micron and d₉₀ of about 4 micron.

Similar to solution-based formulation, for proper deposition of the emitted formulation in the lung epithelium, a preferred range of MMAD is required. In some embodiments, the MMAD of the emitted formulation is between 1 micron and 5 microns. In other embodiments, the MMAD of the emitted formulation is between 2 microns and 3 microns.

Stability of a suspension-based formulation can be determined by a variety of methods. One such method is to measure the fine particle dose (FPD) over time in different temperature/humidity conditions. Typically, the formulation will see an initial drop in FPD, but the FPD should remain substantially unchanged after this initial drop if the aerosol formulation is stable. In contrast, in an unstable aerosol formulation, and therefore undesirable, the FPD will continue to decrease over time. A stable aerosol formulation will have a FPD that remain substantially unchanged after the initial drop at conditions of 25° C. and 60% relative humidity (RH) for at least 3 weeks after formulation, at least 4 weeks after formulation, at least 5 weeks after formulation, at least 6 weeks after formulation, at least 8 weeks after formulation, at least 10 weeks after formulation, at least 12 weeks after formulation, or at least 6 months after formulation. In other embodiments, a stable aerosol formulation will have a FPD that remain substantially unchanged after the initial drop at accelerated conditions of 40° C. and 75% relative humidity (RH) for at least 3 weeks after formulation, at least 4 weeks after formulation, at least 5 weeks after formulation, at least 6 weeks after formulation, at least 8 weeks after formulation, at least 10 weeks after formulation, at least 12 weeks after formulation or at least 6 months after formulation.

Another measure of stability for suspension-based aerosol formulations is the measure of the mass median aerodynamic diameter (MMAD) of the particles overtime. Typically, the formulation will initially see an increase in MMAD, but then the MMAD should plateau (coinciding with a stabilization of FPD) after this initial increase. A stable aerosol formulation will have a MMAD that remain substantially unchanged after the initial increase at conditions of 25° C. and 60% relative humidity (RH) for at least 3 weeks after formulation, at least 5 weeks after formulation, at least 6 weeks after formulation, at least 8 weeks after formulation, at least 10 weeks after formulation, at least 12 weeks after formulation, or at least 6 months after formulation. In other embodiments, a stable suspension-based aerosol formulation will have a MMAD that remain substantially unchanged after the initial increase at acceleration conditions of 40° C. and 75% relative humidity (RH) for at least 3 weeks after formulation, at least 4 weeks after formulation, at least 5 weeks after formulation, at least 6 weeks after formulation, at least 8 weeks after formulation, at least 10 weeks after formulation, at least 12 weeks after formulation or at least 6 months after formulation.

### Controlled-Release Rotigotine Formulations

Compared to contention oral route (tablet, pill, capsule, etc.) a key benefit for respiratory delivery of rotigotine is avoiding first pass metabolism. By avoiding first pass metabolism, this enhances the bioavailability of rotigotine significantly. The oral inhalation delivery route can also provide rapid onset of action and therefore can be ideal for formulations intended for acute rescue therapy. However, there exists a significant need to expand the role of oral inhalation aerosols as controlled-release therapy for various indications. In one embodiment, the invention is related to method to deliver a controlled-release rotigotine formulation to a patient in need of such therapy by oral inhalation in order to treat one or more symptoms of Parkinson's disease. A challenge with controlled-release formulation administered via oral inhalation is that the nature of the lungs provides a rapid clearance mechanism for foreign particles. Several methodologies are described below to accomplish a controlled-release rotigotine formulation for administration through oral inhalation for treatment of one or more symptoms of Parkinson's disease.

One method for formulating a controlled-release medicament is to encapsulate the active pharmaceutical ingredient (in the case of the present invention, rotigotine) in a slow-degrading polymer matrix. This provides the opportunity for controlled-release effects through polymer degradation and diffusion through the polymeric matrix. There are many suitable polymeric matrix components including but not limited to poly(lactic-co-glycolic acid) or PLGA, polylactic acid or PLA, polycaprolactone or PCL and their derivatives, cellulose, albumin, sodium hyaluronate, polyanhydrides, poly(vinyl acetate) or PVA, polyethylene glycol or PEG and derivatives, chitosan, hyaluronic acid, sodium alginate, starch, oligo/polysaccharides or any of the foregoing in combination.

Another method for formulating a controlled-release rotigotine is to chemically conjugate rotigotine to a dendrimer or hyperbranched polymer. By covalently bonding conjugation, rotigotine can be attached to a dendrimer or hyperbranched polymer at the molecular level through reaction between hydroxyl groups in the presence of an appropriate linker molecule and catalyst. The obtained rotigotine conjugated dendrimer or hyperbranched polymer is expected to achieve a controlled-release effect by the cleavage of rotigotine from the hyperbranched polymer or dendrimer by enzymes that are naturally found in the lungs (de-esterification). Similar to this concept, chemical conjugation to other carrier molecules such as dextran, polyethylene glycol, oleic acid, palmitic acid and stearic acid can also provide a controlled-release or sustained release effect.

Another method for formulating a controlled-release or sustained release rotigotine is especially applicable for a formulation that is suitable for administration using a pressurized metered dose inhaler (pMDI). This methodology consists of dissolving an HFA-soluble excipient such as PEG-PLA copolymer, sugar acetate and PLA into HFA-based rotigotine MDI suspension formulation. Once actuated from the canister as an aerosol, the excipients solubilized in HFA would stick to the rotigotine particles to produce a coated particle in-situ as the HFA propellant evaporates. The excipient coating on the rotigotine particles provides a controlled-release or sustained release effect in the lungs where the API is deposited after inhalation.

Another method for formulating a controlled-release or sustained release rotigotine is the use of a solid lipid nanoparticle or SLN. A solid lipid nanoparticle is typically spherical with an average diameter between 10 to 1000 nanometers. Solid lipid nanoparticles possess a solid lipid core matrix that can solubilize lipophilic molecules. The lipid core is stabilized by surfactants or emulsifiers. Rotigotine can be solubilized within the solid lipid core of the SLN and slowly released from the SLN in the lungs to provide a controlled-release or sustained release effect.

There are various techniques to prepare the physical encapsulated or chemical conjugated rotigotine-containing particles. These techniques include but are not limited to emulsion-based techniques, spray-drying, freeze drying, super critical fluid based techniques, solvent/anti-solvent precipitation, homogenization/grinding, etc. The rotigotine particles can also have a variety of different shapes or morphologies as long as the size/shape is uniform throughout the particle population. These shapes or morphologies include sphere, flake, spindle, needle, cube, and square-bifrustum. In some embodiments, the rotigotine particles of the invention have a mean diameter from about 100 nm to about 10 microns. In other embodiments, the rotigotine particles of the invention have a mean diameter from about 1 micron to about 3 micron.

### III. ORAL INHALATION DELIVERY SYSTEMS

The preferred embodiment of the rotigotine is delivered using inhalation therapy. Many preclinical and clinical studies with inhaled compounds have demonstrated that efficacy can be achieved both within the lungs and systemically. Moreover, there are many advantages associated with pulmonary delivery including rapid onset, the convenience of patient self-administration or with minimal assistance from a second person, the potential for reduced drug side-effects, ease of delivery by inhalation, the elimination of needles, and the like.

A wide variety of delivery methods/platforms are suitable for the practice of the invention Inhalation devices or other non-injectable devices are preferred devices and function by delivering an aerosol of the drug formulation into the subject or patient. These inhalation devices generally including a housing having a proximal end and a body portion. A mouthpiece or nose piece will typically be positioned at the proximal end.

### Nebulizers

Nebulizers generate an aerosol from a liquid, some by breakup of a liquid jet and some by ultrasonic vibration of the liquid with or without a nozzle. Liquid formulations are prepared and stored under aseptic or sterile conditions since they can harbor microorganisms. Liquid formulation can either be a suspension formulation or a solution formulation. The use of preservatives and unit dose packaging is contemplated. Additionally, solvents, detergents and other agents can be used to stabilize the drug formulation.

In general, drug compositions for oral inhalation delivery using nebulizers are aqueous solutions, dispersions or suspensions that are aerosolized and then inhaled. The aerosol comprises very fine droplets of the drug compositions dispersed in air. The droplets are necessarily less than about five microns in geometric diameter to provide respirable droplets that enable delivery of the aerosolized drug formulation to the respiratory tract beyond the oropharynx upon inhalation. This process is called atomization. Typically, the drug composition contains particles of the therapeutic agent(s) or a solution of the therapeutic agent(s), and any necessary excipients. The droplets carry the therapeutic agent(s) into the nose, upper airways or deep lung when the aerosol cloud is inhaled by the patient or subject.

Aerosol generators, or nebulizers, apply mechanical shearing forces to the drug formulation by various means to break up the formulation surface or generate filament streams to form the droplets. Nebulizers typically use pneumatic, piezoelectric, ultrasonic, or electromechanical means to generate shearing forces. The nebulizers may also incorporate baffling mechanisms to remove larger, non-respirable droplets from the aerosol. In use, the nebulized drug formulation is administered to the individual or subject via a mouthpiece or mask.

Traditional nebulizer devices, such as pneumatic or jet nebulizers, are commonly used for drug formulation delivery and are suitable for use in the present invention. Pneumatic (jet) nebulizers use a pressurized gas supply as a driving force for liquid atomization. Compressed gas is delivered through a nozzle or jet to create a low pressure field which entrains a surrounding bulk liquid or drug composition and shears it into a thin film or filaments. The film or filaments are unstable and break up into small droplets which are carried by the compressed gas flow into the inspiratory breath. Sometimes baffles are inserted into the droplet plume in order to screen out the larger droplets and return them to the bulk liquid reservoir. However, one drawback of pneumatic nebulizers is that these devices require extended administration time, lasting up to 30 minutes, which often results in low patient compliance. In addition, the uniformity of the delivered dose of drug formulation from jet nebulizers can be challenging especially for suspension-based formulations.

A particular group of nebulizers, referred to herein as “next generation nebulizers”, use meshes or membranes to produce fine droplet sprays. These devices are much more efficient at producing aerosols, and can significantly reduce administration time. the meshes/membranes in next generation nebulizers contain many apertures or pores that have diameters typically between 1 and 8 microns. The drug formulation is forced through the mesh apertures by piezoelectric or electromechanical “pumping” or, alternatively, the mesh is vibrated to reciprocate through a pool of the drug formulation, thereby generating multiple liquid filaments with diameters approximating the mesh apertures. The filaments breakup to form droplets with diameters approximating the diameters of mesh apertures. These next generation nebulizers are efficient aerosol generators and they minimize the duration of administration. This is because next generation nebulizers can form aerosols that have a high proportion of respirable aerosol droplets, including those with diameters much less than 4.7 microns mass median aerodynamic diameter (MMAD per compendial method USP 601), which enables quick and efficient delivery of the aerosolized drug to the respiratory tract.

### Pressurized Metered Dose Inhalers

Pressuried metered dose inhalers or pMDIs, are an additional class of aerosol dispensing devices. pMDIs package the API formulation in a canister under pressure with a solvent and propellant mixture. Upon dispensed a jet of the mixture is ejected through a valve and nozzle and the propellant “flashes off” leaving an aerosol of the API formulation.

Propellants may take a variety of forms. In a non-limiting example, the propellant may be a compressed gas or a liquefied gas. Chlorofluorocarbons (CFCs) were once commonly used as liquid propellants, but have now been banned due to the negative impact on the earth's ozone layer. They have been replaced by the now widely accepted hydrofluorocarbon or hydrofluoroalkane (HFA) propellants. The most commonly used HFAs are 1,1,1,2-tetrafluoroethane, which is also referred to as 134a or HFA 134a; and 1,1,1,2,3,3,3-heptafluoropropane, which is also referred to as 227 or HFA 227, both available from Dupont, Solvay Chemicals, or Mexichem Fluor. In some cases, the propellant can be one HFA compound or a mixture of two or more HFA compounds.

In some embodiments, the propellant is selected from the group consisting of 1,1,1,2-tetrafluoroethane and 1,1,1,2,3,3,3-heptafluoropropane. In other embodiments, the propellant is 1,1,1,2-tetrafluoroethane. In some embodiments, the propellant is 1,1,1,2,3,3,3-heptafluoropropane. In some embodiments, the propellant is a mixture of 1,1,1,2-tetrafluoroethane and 1,1,1,2,3,3,3-heptafluoropropane.

The canister may contain multiple doses of the drug composition, although it is possible to have single dose canisters as well. The canister may include a valve, from which the contents of the canister may be discharged. In some embodiments, the valve is a metering valve. Aerosolized drug composition is dispensed from the pMDI by applying a force on the canister to push it into the receptacle, thereby opening the valve and causing the drug particles to be conveyed from the valve through the receptacle outlet. Upon discharge from the canister, the drug composition particles are atomized, forming an aerosol. pMDIs generally use propellants to pressurize the content of the canister and to propel the drug particles out of the receptacle outlet. In pMDIs, the drug composition is provided in liquid form, and resides within the canister along with the propellant.

In some instances, a manual discharge of aerosolized drug must be coordinated with inhalation, so that the drug composition particles are entrained within the inspiratory air flow and conveyed to the lungs. In other instances, a breath-actuated trigger, such as that included in the Tempo® inhaler (Allergan, Inc., Irvine, Calif.) may be employed that simultaneously discharges a dose of drug upon sensing inhalation. Such breath-actuated pMDI automatically discharges the drug composition aerosol at the appropriate time during inhalation by the user or subject. These devices are generally known as breath-actuated pressurized metered dose inhalers. Additionally, along with breath actuation, these devices may also be breath synchronized so as to discharge the bolus of the formulation at the height (largest volume) of the inspiratory breath. Breath-actuated pMDIs have additionally advantages including enhanced patient compliance and efficient, reliable dose-to-dose consistency that is independent of the inhalation flow rate. For example, the Tempo inhaler achieves these advantages by combining proprietary features such as a breath synchronized trigger and the flow control chamber and dose counter/lockout in a small, easy to use device. These advanced aerodynamic control elements are driven only by the patient's breath, avoiding expensive, power consuming electronics, resulting in an affordable, reliable, easy to use, and disposable platform. Because of the small size and option of breath-synchronization, a patient with Parkinson's disease is able to operate the device either to self-administer the formulation when needed, or with relatively little assistance from a second person who does not need to have formal medical training. In some variations, the pMDI can be fitted with a face piece or other adaptor to administer the drug for better and/or more efficient delivery.

### Dry Powder Inhalers

In a dry powder inhaler (DPI) the dose of the drug composition to be administered is stored in the form of a dry powder and on actuation of the inhaler, the particles of the powder are inhaled by the subject. Similar to pressurized metered dose inhalers (pMDIs), a compressed gas may be used to dispense the powder. Alternatively, when the DPI is breath-actuated (and therefore does not use a compressed gas to dispense the powder), the powder may be packaged in various forms such as a loose powder, cake or pressed shape in a reservoir. Non-limiting examples of these breath-actuated DPIs include the Turbohaler™ inhaler (AstraZeneca, Wilmington, Del.) and Clickhaler® inhaler (Innovata, Ruddington, Nottingham, UK). In some cases, for DPIs, a doctor blade or shutter slides across the powder, cake or pressed shape and the powder is culled into a flowpath whereby the subject can inhale the powder in a single breath. Other powders are packaged as blisters, gelcaps, tabules, or other preformed vessels that can be pierced, crushed or otherwise unsealed to release the powder into the flowpath for subsequent inhalation. Still other DPIs release the powder into a chamber or capsule and use mechanical or electrical agitators to keep the drug suspended for a short period until the patient inhales.

All references cited herein are incorporated by reference in their entireties, whether previously specifically incorporated or not. The publications mentioned herein are cited for the purpose of describing and disclosing reagents, methodologies and concepts that may be used in connection with the present invention. Nothing herein is to be construed as an admission that these references are prior art in relation to the inventions described herein.

Although this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

The following examples serve to more fully describe and exemplify the above disclosed embodiments. It is understood that these examples in no way serve to limit the true scope of this disclosure, but rather are presented for illustrative purposes.

### IV. EXAMPLES

### Example 1

### In Situ Rotigotine Salt Screening

Rotigotine, bulk drug substance, was purchased from Chemagis (Perrigo API). A stock solution of rotigotine was prepared by dissolving rotigotine particles in ethanol at 10 mg/mL. With 1.5 mL rotigotine stock solution added to formulation bottles, each formulation bottle contained 15 mg rotigotine. For preparation of acid stock solutions, it was assumed that complete reaction or ion-pairing between rotigotine and acid. Based on the molecular weight and number of anions of each acid as listed in Table 1, acids stock solutions were prepared by dissolving the required amount in ethanol.

Each formulation bottle was filed with 1.5 mL of rotigotine stock solution and 1.0 ml, of acid stock solution. The formulation bottles (PET bottles) were then sealed with continuous valves and vortexed for 30 seconds to mix the solution well. Finally, 17.6 g of 1,1,1,2,3,3,3-heptafluoropropane (HFA227 (Mexichem)) to make a final volume of 15 mL of rotigotine formulation. All the bottles were hand shaken for 10 seconds.

All in situ salt screening samples were made as 1 mg/mL of rotigtine free base in 10% (w/w) ethanol in HFA 227. Acids corresponding to the salt forms in Table 1 were added according to the molar ratio with rotigotine for complete reaction. As 1 mg/mL was the minimal required dose at the time of experiment, all formulations were tested at this level to screen out non-solution salt formulations.

Among the acids tested for in situ salt screening, 5 formed white precipitate immediately upon preparation. These were the rotigotine citrate, fumarate, succinate, sulfate, tartrate, xinafoate salt formulations. These 5 rotigotine salt formulations were immediately ruled out as the precipitate formation was not desired. All clear solution samples were stored at room temperature (RT) for 1 week. After 1 week, 3 formed red precipitate. These were rotigotine acetate, benzoate and mesylate salt formulations. These 3 rotigotine salt formulations were also ruled out as precipitate formation is not desired. The rest of the samples remained as clear solutions (stable) at after another week at 5° C.

Out of the 7 acids that remained a clear solution after 1 week of storage, rotigotine maleate was selected as the lead candidate and suitable as a stable solution for oral pulmonary aerosol delivery. The selection criteria was based on the observation that the rotigotine maleate solution stayed in solution formulation at the minimum required concentration (1 mg/mL) and the maleate salt has been used in approved inhaled products, such as the Neohaler™.

TABLE 1
In situ screening list of rotigotine salts.

| | | | | Observation | | |
|---|---|---|---|---|---|---|
| | | MW | | | 1 week at | 1 week at |
| Salt | Anion | (g/mol) | Structure | Immediate | RT | 5° C. |
| Acetate | 1 | 60 | 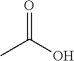 | solution | red precipitate | N/A |
| Benzoate | 1 | 122 | 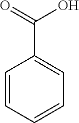 | solution | red precipitate | N/A |
| Citrate | 3 | 192 | 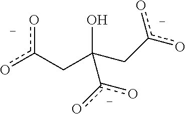 | white precipitate | N/A | N/A |
| Fumarate | 2 | 116 | 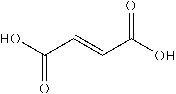 | white precipitate | N/A | N/A |
| Glycolate | 1 | 76 | 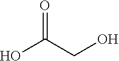 | solution | solution | solution |
| Lactate | 1 | 90 | 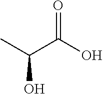 | solution | solution | solution |
| Maleate | 2 | 116 | 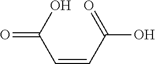 | solution | solution | solution |
| Mesylate | 1 | 96 | 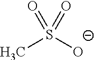 | solution | red precipitate | N/A |
| Palmitate | 1 | 284 | 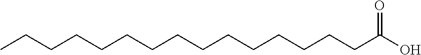 [SMILES: CCCCCCCCCCCCCCCC(=O)O] | solution | solution | solution |
| Pamoate | 2 | 388 | 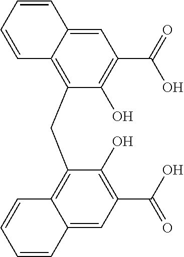 [SMILES: O=C(O)c1cc2ccccc2c(Cc2c(O)c(C(=O)O)cc3ccccc23)c1O] | solution | solution | solution |
| Propionate | 1 | 74 | 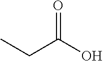 | solution | solution | solution |
| Stearate | 1 | 256 | 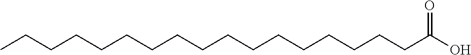 [SMILES: CCCCCCCCCCCCCCCCCC(=O)O] | solution | solution | solution |
| Succinate | 2 | 118 | 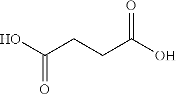 | white precipitate | N/A | N/A |
| Sulfate | 2 | 98 | 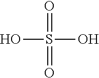 | white precipitate | N/A | N/A |
| Tartrate | 2 | 150 | 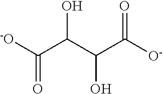 | white precipitate | N/A | N/A |
| Xinafoate | 1 | 188 | 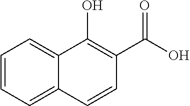 [SMILES: O=C(O)c1ccc2ccccc2c1O] | White precipitate | N/A | N/A |

We claim:

1. A pharmaceutical formulation comprising a dopamine agonist, wherein the pharmaceutical formulation is suitable for administration by oral inhalation.

2. The pharmaceutical formulation of claim 1 wherein the dopamine agonist is rotigotine or a pharmaceutically acceptable salt thereof.

3. The pharmaceutical formulation of claim 2 wherein the formulation is administered by oral inhalation using a device selected from the group consisting of a nebulizer, a pressurized metered dose inhaler, a dry powder inhaler.

4. The pharmaceutical formulation of claim 3, wherein the device is a pressurized metered dose inhaler and the formulation further comprises a propellant.

5. The pharmaceutical formulation of claim 4, wherein the propellant is 1,1,1,2-tetrafluoroethane, 1,1,1,2,3,3,3-heptafluoropropane, or a mixture thereof.

6. The pharmaceutical formulation of claim 5, wherein the formulation is a solution-based formulation or a suspension-based formulation.

7. The pharmaceutical formulation of claim 6, wherein the formulation is a suspension-based formulation and the size distribution of the rotigotine particles has a d₁₀ of about 0.5 micron to about 1.0 micron, d₅₀ of about 1 micron to about 2 micron and d₉₀ of about 2 micron to about 3 micron.

8. The pharmaceutical formulation of claim 6, wherein the formulation is a suspension-based formulation and the size distribution of the rotigotine particles has a d₁₀ of about 1 micron, d₅₀ of about 2 micron to 3 micron and d₉₀ of about 4 micron.

9. The pharmaceutical formulation of claim 2, wherein the formulation is suitable for controlled-release or sustained-release of rotigotine in the lungs after administration by oral inhalation.

10. The pharmaceutical formulation of claim 9, wherein the rotigotine is physically encapsulated into a polymeric excipient.

11. The pharmaceutical formulation of claim 10, wherein the polymeric excipient is selected from the group consisting of poly(lactic-co-glycolic acid), polylactic acid, polycaprolactone, cellulose, albumin, sodium hyaluronate, polyanhydrides, poly(vinyl acetate), polyethylene glycol, chitosan, hyaluronic acid, sodium alginate, starch, oligosaccharides and polysaccharides.

12. The pharmaceutical formulation of claim 9, wherein the rotigotine is chemically conjugated to a carrier selected from the group consisting of a dendrimer, a hyperbranched polymer, polyethylene glycol, dextran, oleic acid, palmitic acid, and stearic acid.

13. The pharmaceutical formulation of claim 9, wherein the rotigotine is encapsulated in a solid lipid nanoparticle.

14. The pharmaceutical formulation of claim 4, wherein the formulation further comprises an excipient selected from the group consisting of polyethylene glycol-polylactic acid copolymer, a sugar acetate, and polylactic acid.
